(19) United States
(12) Patent Application Publication
(10) Pub. No.: US 2024/0050348 A1
(43) Pub. Date: Feb. 15, 2024

(54) Hair and Scalp Compositions and Related Treatment Methods

(71) Applicants: Alan Joel Meyers, Boca Raton, FL (US); Douglas Charles Tomczak, Coral Gables, FL (US); Marianne Cliville, Glen Ridge, NJ (US)

(72) Inventors: Alan Joel Meyers, Boca Raton, FL (US); Douglas Charles Tomczak, Coral Gables, FL (US); Marianne Cliville, Glen Ridge, NJ (US)

(21) Appl. No.: 18/231,599

(22) Filed: Aug. 8, 2023

Related U.S. Application Data
(60) Provisional application No. 63/397,195, filed on Aug. 11, 2022.

(51) Int. Cl. A61K 8/49; A61K 8/34; A61Q 7/00

(52) U.S. Cl. CPC A61K 8/4953; A61K 8/492; A61K 8/347; A61Q 7/00; A61K 2800/5922; A61K 2800/81

(57) ABSTRACT

A topically applied scalp treatment composition and related method is provided including caffeine, melatonin, a meroterpene and a cosmetically acceptable carrier, and wherein the composition treated scalp area has an anagen to telogen ratio increase from a baseline control of 3.8 compared to at least about 4.6 units of ratio after a 30-day daily application to the scalp area.

### BACKGROUND OF THE INVENTION

### Field of the Invention

The invention relates to cosmetic compositions for topical application to mammalian hair and scalp capable of promoting hair growth.

### The Related Art

In most mammals hair does not grow continuously but rather undergoes a cycle of activity involving alternate periods of growth and rest. The hair growth cycle can be divided into three main stages, namely:
(i) the growth phase known as anagen, during which the hair follicle penetrates deep into the dermis with cells of the bulb dividing rapidly and differentiating to form the hair,
(ii) the transitional stage known as catagen, which is heralded by the cessation of mitosis, and during which the follicle regresses upwards through the dermis and hair growth ceases,
(iii) the resting stage known as telogen, in which the regressed follicle contains a small secondary germ with an underlying ball of tightly packed dermal papilla cells.

The initiation of a new anagen phase is revealed by rapid proliferation of epithelial cells in the germ, expansion of the dermal papilla and elaboration of basement membrane components. The hair cycle is then repeated many times until, as a consequence of onset of male pattern baldness, most of the follicles spend an increasing proportion of their time in the telogen stage, and the hairs produced become finer, shorter, and less visible; this is known as terminal to vellus transformation.

The art describes many compounds and compositions allegedly capable of curing baldness or at least minimizing hair loss. With one exception, none of the reported hair treatments have achieved commercial success, no doubt due to ineffectiveness. An exception is minoxidil, sold under the mark Rogaine®.

An objective of the present invention was to identify compounds and compositions that could reawaken anagen to stimulate vellus transformation into new hair growth, slow rate of hair loss, thicken hair and/or remove oxidative stress against hair formation.

### SUMMARY OF THE INVENTION

A topically applied scalp treatment composition is provided including:
(i) about 0.001 to about 1% by weight of the composition of caffeine;
(ii) about 0.0001 to about 1% by weight of the composition of melatonin;
(iii) about 0.001 to about 3% by weight of the composition of a meroterpene; and
(iv) about 0.1 to about 99% by weight of the composition of a cosmetically acceptable carrier, and wherein the treatment composition when applied to a scalp area induces an anagen to telogen ratio increase from a baseline control of about 3.8 compared to at least about 4.6 units of ratio after a 30-day daily application.
   Advantageously, the treatment composition when applied to a scalp area induces an anagen to telogen ratio increase from a baseline control of about 3.5 compared to at least about 5.1 units of ratio after a 60-day daily application.

Preferably the meroterpene is bakuchiol. Other useful components of the composition are resveratrol and red clover extract.

Also disclosed herein are methods of use utilizing the compositions. These methods relate to hair growth promotion, hair loss inhibition, thickening of hair, stress protection of hair and scalp, and combinations thereof.

### DETAILED DESCRIPTION OF THE INVENTION

Herein we disclose formulations having ability to stimulate growth of hair on mammalian scalp. These formulations host melatonin, caffeine and meroterpene.

Melatonin is found in plants and animals. It is primarily known in animals as a hormone released by the pineal gland in the brain at night, and has long been associated with control of the sleep-wake cycle. In vertebrates, melatonin is involved in synchronizing circadian rhythms, including sleep-wake timing and blood pressure regulation, and in control of seasonal rhythmicity including reproduction, fattening, moulting and hibernation. Many of its effects are through activation of the melatonin receptors, while others are due to its role as an antioxidant. In plants, it functions to defend against oxidative stress. It is also present in various foods.

Melatonin has an IUPAC name which is N-[2-(5-methoxy-1H-indol-3-yl) ethyl]acetamide.

Compositions reported herein may contain melatonin in amounts from about 0.0001 to about 2%, particularly from about 0.001 to about 1%, most especially from about 0.0015 to about 0.005%, by weight of the composition.

Caffeine is a central nervous system (CNS) stimulant of the methylxanthine class. There are several known mechanisms of action to explain the effects of caffeine. The most prominent is that it reversibly blocks the action of adenosine on its receptors and consequently prevents the onset of drowsiness induced by adenosine. Caffeine also stimulates certain portions of the autonomic nervous system. It is also used as a cognitive enhancer which increases alertness and attentional performance.

Caffeine is a bitter, white crystalline purine, a methylxanthine alkaloid, and is chemically related to the adenine and guanine bases of deoxyribonucleic acid (DNA) and ribonucleic acid (RNA). It is found in the seeds, fruits, nuts, or leaves of a number of plants native to Africa, East Asia and South America, and helps to protect them against herbivores and from competition by preventing the germination of nearby seeds as well as encouraging consumption by select animals such as honey bees. The best-known source of caffeine is the coffee bean, the seed of the Coffea plant. People may drink beverages containing caffeine to relieve or prevent drowsiness and to improve cognitive performance. To make these drinks, caffeine is extracted by steeping the plant product in water, a process called infusion.

Caffeine has the IUPAC name of 1,3,7-Trimethylpurine-2,6-dione.

Compositions reported herein may contain caffeine in amounts from about 0.0001 to about 2%, particularly from about 0.001 to about 1%, most especially from about 0.05 to about 0.1%, by weight of the composition.

Meroterpenes are chemical compounds having a partial terpenoid structure. Examples include bakuchiol, ferruginol, mutisianthol and totarol. Most preferred is bakuchiol, particularly s-bakuchiol.

Compositions reported herein may contain meroterpenes in total amounts from about 0.0001 to about 2%, particularly from about 0.01 to about 1%, most especially from about 0.05 to about 0.5%, by weight of the composition.

Resveratrol (chemically described as 3,5,4′-trihydroxy-trans-stilbene) is a stilbenoid, a type of natural phenol, and a phytoalexin produced by several plants in response to injury or when the plant is under attack by pathogens, such as bacteria or fungi. Sources of resveratrol in food include the skin of grapes blueberries, raspberries, mulberries, and peanuts.

Compositions reported herein may contain resveratrol in amounts from about 0.0001 to about 2%, particularly from about 0.001 to about 1%, most especially from about 0.05 to about 0.1%, by weight of the composition.

Compositions used to treat a scalp area will have an anagen to telogen ratio increase from a baseline control of about 3.8 compared to at least about 4.6 after a 30-day daily placement on the scalp area. Advantageously, the compositions described herein treat the scalp area having hair characterized by anagen to telogen ratio increasing from a baseline control value of about 3.5 compared to at least about 5.1 after a 60-day daily placement of treatment composition onto the scalp.

Compositions of the present disclosure are generally formulated with a cosmetically acceptable carrier. Amounts of the carrier may range from 1 to 99.9%, preferably from 70 to 95%, optimally from 80 to 90% by weight of the composition. Among the useful carriers are water, oils/emollients, gels, fatty acids, fatty alcohols, humectants, thickeners and combinations thereof. The carrier may be aqueous, anhydrous or an emulsion. Preferably the compositions are aqueous, especially water and oil emulsions of the W/O or O/W or triplex W/O/W variety. Water when present may be in amounts ranging from 5 to 98%, preferably 20 to 70%, optimally from 35 to 60% by weight.

Water, when present as carrier or otherwise may advantageously be incorporated into the compositions as a deionized, sterilized or pasteurized liquid or can be heat treated or irradiated after having been mixed with other components of the composition. These treatments ensure elimination of pathogenic microbes.

Oils/Emollients

Oils/Emollients may serve as physiologically acceptable carriers. These may be in the form of silicone oils, synthetic or natural esters and hydrocarbons. Amounts of the emollients may range anywhere from 0.1 to 95%, preferably between 1 and 50% by weight of the cosmetic product.

Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 4 to 5, silicon atoms.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes with viscosities of from about 5×10⁻⁶ to 0.1 m²/s at 25° C. Among the preferred nonvolatile emollients useful in the present compositions are the polydimethyl siloxanes having viscosities from about I×10⁻⁵ to about 4×10⁻⁴ m²/s at 25° C.

Another class of nonvolatile silicones are emulsifying and non-emulsifying silicone elastomers. Representative of this category is Dimethicone/Vinyl Dimethicone Crosspolymer available as Dow Corning 9040, General Electric SFE 839, and Shin-Etsu KSG-18. Silicone waxes such as Silwax WS-L (Dimethicone Copolyol Laurate) may also be useful.

Among the ester oils/emollients are:
a. Alkyl esters of saturated fatty acids having 10 to 24 carbon atoms. Examples thereof include behenyl neopentanoate, isononyl isonanonoate, isopropyl myristate and octyl stearate.
b. Ether-esters such as fatty acid esters of ethoxylated saturated fatty alcohols.
c. Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monostearate, ethoxylated propylene glycol monostearate, glyceryl mono- and di-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C1-C30 alcohols.
d. Wax esters such as beeswax, spermaceti wax and tribehenin wax.
e. Sugar ester of fatty acids such as sucrose polybehenate and sucrose polycottonseedate.

Hydrocarbons which are suitable carriers include petrolatum, mineral oil, isoparaffins, and especially isohexadecane, available commercially as Permethyl 101 A from Presperse Inc.

Fatty acids having from 10 to 30 carbon atoms may also be suitable as carriers. Illustrative of this category are pelargonic, lauric, myristic, palmitic, stearic, isostearic, oleic, and behenic acids.

Fatty alcohols having from 10 to 30 carbon atoms are another useful category of carrier. Illustrative of this category are stearyl alcohol, lauryl alcohol, myristyl alcohol and cetyl alcohol.

Further oils/esters include hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, coriander oil, castor oil, tea tree oil, avocado oil, caprylic/capric acid triglycerides, for instance those sold by the company Stearineries Dubois or those sold under the names Miglyol 810, 812 and 818 by the company Dynamit Nobel, jojoba oil, shea butter oil and caprylyl glycol; synthetic esters and ethers, especially of fatty acids, for instance Purcellin oil, 2-octyldodecyl stearate, 2-octyldodecyl erucate, isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate, or isopropyl lauroyl sarcosinate, sold especially under the trade name Eldew SL 205 by the company Ajinomoto; linear or branched hydrocarbons, of mineral or synthetic origin, such as volatile or non-volatile liquid paraffins, and derivatives thereof, petroleum jelly, polydecenes, isohexadecane, isododecane, hydrogenated polyisobutene such as Parleam oil, or the mixture of n-undecane (C₁₁) and of n-tridecane (C₁₃) sold under the reference Cetiol UT by the company Cognis.

Additional examples include benzoic acid esters of C₉-C₁₅ alcohols, isononyl iso-nonanoate, C₁₂-C₁₅ alkyl benzoate, or any combinations thereof.

Examples of amphiphilic organic solvents include: polypropylene glycol (PPG) like propylene glycol alkyl ester or alkyl ether of PPG like PPG-23 oleyl ether and PPG-36 oleate.

The above lists are only examples and not limiting. The total amount of oils/emollients present in the compositions is typically about 0.1, 0.5, 1.0, or 2.5 wt. % to about 5.0, 7.5, 10.0, 15.0, 20.0, or 30 wt. % of the total weight of the composition.

Humectants

Humectants of the polyhydric alcohol-type can be employed as carriers. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol, C1-C8 alcohols, dialkyl isosorbides such as dimethyl isosorbide and mixtures thereof. Yeast extracts (Saccharomyces cerevisiae) can also deliver soothing humectancy. The amount of humectant may range anywhere from 0.5 to 50%, preferably between 1 and 15% by weight of the composition.

Polyglutamic acid and salts thereof such as sodium polyglutamate, sourced under the trademark Gamma-Max, are moisture capture and retention agents which reduce transdermal water loss from scalp and hair. Amounts may range from about 0.01 to about 10% by weight of the composition.

Besides providing humectancy, certain polyols perform as skin penetration enhancers. These serve to facilitate movement of actives into scalp and hair. Particularly useful are propanediols, especially 1,2-propanediol. Amounts may range from about 0.1 to about 20%, especially from about 0.5 to about 10% by weight of the compositions.

Peptides and Plant Extracts

Certain peptides are useful in controlling hair loss, stimulating hair growth, and reducing inflammation of the scalp. Of particular effectiveness is acetyl tetrapeptide-3. Enhancement of activity may be achieved by combination with red clover flower extract. The combination of acetyl tetrapeptide-3 and red clover flower extract can be commercially sourced under the trademark Capixyl from Lucas Meyer Company. Amounts of the peptide/red clover extract may range from about 0.1 to about 5%, especially from about 0.5 to about 3% by weight of the composition.

Another useful plant extract is Picea mariana Bark extract, taken from black spruce bark. This protects hair color against external stresses, repeated washes, and UV induced discoloration. The extract is sold under the trademark Borealine Protect by Lucas Meyer Company. Amounts may range from about 0.01 to about 1%, especially from about 0.05 to about 0.5% by weight of the composition.

Pisum sativum (Pea) extract may help hair defense by assisting scalp to limit hair cell death in stress conditions. Commercially, this extract in water is sold under the trademark Procataline by ASI/ISP. Amounts may range from about 0.1 to about 2%, especially from about 0.1 to about 1% by weight of the composition.

Thickeners

Thickeners can be utilized as part of the dermatologically acceptable carriers. Typical thickeners include crosslinked acrylates (e.g. Carbopol 982®), hydrophobically-modified acrylates (e.g. Carbopol 1382®), cellulosic derivatives and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums may be suitable thickeners and can include guar, xanthan, sclerotium, carrageenan, pectin and combinations of these gums. Inorganics may also be utilized as thickeners, particularly clays such as bentonites and hectorites, fumed silicas, and silicates such as magnesium aluminum silicate (Veegum®). Amounts of the thickener may range from 0.0001 to 10%, usually from 0.001 to 1%, optimally from 0.01 to 0.5% by weight of the product.

Organic UV Filters

There may be present organic UV filters. They include both UVA and UVB protective ranges. Organic sunscreens will have at least one chromophoric group absorbing within the ultraviolet ranging from 290 to 400 nm. Chromophoric organic sunscreens may be divided into the following categories (with specific examples) including: p-Aminobenzoic acid, its salts and its derivatives (ethyl, isobutyl, glyceryl esters; p-dimethylaminobenzoic acid); Anthranilates (o-aminobenzoates; methyl, menthyl, phenyl, benzyl, phenylethyl, linalyl, terpinyl, and cyclohexenyl esters); Salicylates (octyl, amyl, phenyl, benzyl, menthyl, glyceryl, and dipropyleneglycol esters); Cinnamic acid derivatives (menthyl and benzyl esters, alpha-phenyl cinnamonitrile; butyl cinnamoyl pyruvate); Hydrocarbons (diphenylbutadiene, stilbene); Dibenzalacetone and benzalacetophenone; Naphtholsulfonates (sodium salts of 2-naphthol-3,6-disulfonic and of 2-naphthol-6,8-disulfonic acids); Dihydroxynaphthoic acid and its salts; o- and p-Hydroxybiphenyldisulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate, and tannate); Quinoline derivatives (8-hydroxyquinoline salts, 2-phenylquinoline); Hydroxy- or methoxy-substituted benzophenones; Uric and vilouric acids; Tannic acid and its derivatives (e.g., hexaethylether); (Butyl carbityl) (6-propyl piperonyl) ether; Hydroquinone; Benzophenones (Oxybenzone, Sulisobenzone, Dioxybenzone, Benzoresorcinol, 2,2′,4,4′-Tetrahydroxybenzophenone, 2,2′-Dihydroxy-4,4′-dimethoxybenzophenone, Octabenzone; 4-lsopropyldibenzoylmethane; Butylmethoxydibenzoylmethane; Etocrylene; and 4-isopropyl-dibenzoylmethane).

Particularly important UV filters are: 2-ethylhexyl p-methoxycinnamate (available as Parsol MCX®), 4,4′-t-butyl methoxydibenzoylmethane (known commonly as Avobenzone, available as Parsol 1789®), octylsalicylate (available as Dermablock OS®), tetraphthalylidene dicamphor sulfonic acid (available as Mexoryl SX®), benzophenone-3 (Oxybenzone) and mixtures.

Gelling Agents

Gelling agents may also be included in the sunscreen containing compositions. Examples of suitable hydrophilic gelling agents include carboxyvinyl polymers such as the Carbopol products (carbomers) and the Pemulen products (acrylate/C10-C30-alkylacrylate copolymer); polyacrylamides, for instance the crosslinked copolymers sold under the names Sepigel 305 (CTFA name: polyacrylamide/C13-14 isoparaffin/Laureth 7) or Simulgel 600 (CTFA name: acrylamide/sodium acryloyldimethyltaurate copolymerfisohexadecane/polysorbate 80) by the company SEPPIC; 2-acrylamido-2-methylpropanesulfonic acid polymers and copolymers, which are optionally crosslinked and/or neutralized, for instance the poly(2-acrylamido-2-methylpropanesulfonic acid) sold by the company Hoechst under the trade name “Hostacerin AMPS” (CTFA name: ammonium polyacryldimethyltauramide); cellulose-based derivatives such as hydroxyethylcellulose; polysaccharides and especially gums such as xanthan gum; and mixtures thereof.

Lipophilic gelling agents (thickeners) that may be mentioned include modified clays such as hectorite and its derivatives, for instance the products sold under the name bentone.

In some instances, the gelling agent is ammonium acryloyldimethyltaurate/steareth-25 methacrylate crosspolymer, commercially available from Clariant under the tradename Aristoflex HMS. Most preferred is hydroxyethyl acrylate/sodium acryloyldimethyl taurate copolymer, sold in a fatty ester/water vehicle under trademark of Sepinov WEO.

The above lists are only examples and not limiting.

The gelling agent is typically used in an amount of about 0.05 to about 1.5% by weight, from about 0.08 to about 1.0% by weight, or about 0.1 to about 0.5% by weight, based on the total weight of the composition.

Skin Lightening and Antiaging Agents

Compositions may include skin lighteners such as placental extract, lactic acid, niacinamide, arbutin, kojic acid, ferulic acid, hydroquinone, resorcinol and derivatives including 4-substituted resorcinols, phenylethyl resorcinol and combinations thereof. Amounts of these substances may range from 0.1 to 10%, preferably from 0.5 to 2% by weight of the product.

Also included may be such materials as turmeric (curcumin), ubiquinone (Co-Enzyme CoQ10), alpha-lipoic acid, ellagic acid, kinetin, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B, Ceramide 6 and Ceramide 7 as well as pseudoceramides are useful. Amounts of these materials may range from 0.000001 to 10%, preferably from 0.0001 to 1% by weight of the composition.

Vitamins and Flavonoids

Cosmetic compositions may include vitamins. Illustrative vitamins are Vitamin A (retinol), Vitamin B₂, Vitamin B₃ (niacinamide), Vitamin B₅ (pantothenic acid), Vitamin B₆, Vitamin B12, Vitamin C, Vitamin D, Vitamin E, Vitamin K and Biotin. Derivatives of the vitamins may also be employed. For instance, Vitamin C derivatives include sodium ascorbyl phosphate, ascorbyl tetraisopalmitate, magnesium ascorbyl phosphate and ascorbyl glycoside (Vitamin CG). Derivatives of Vitamin E include tocopheryl acetate, tocopheryl palmitate and tocopheryl linoleate. DL-panthenol and derivatives may also be employed. A particularly suitable Vitamin B₆ derivative is Pyridoxine Palmitate. Flavonoids may also be useful, particularly glucosyl hesperidin, rutin, and soy isoflavones (including genistein, daidzein, equol, and their glucosyl derivatives and mixtures thereof. Also useful are carotenoids such as lycopene (available from watermelon extracts).

Total amount of vitamins or flavonoids when present may range from 0.0001 to 10% by weight of the composition.

The compositions may be formulated into a wide variety of product types that include but are not limited to solutions, suspensions, lotions, creams, gels, toners, sticks, sprays, ointments, cleansing liquid washes and solid bars, shampoos and hair conditioners, hair colorants, pastes, foams, powders, mousses, wipes, hydrogels, film-forming products, facial and skin masks.

Cationic Polymers

Cationic polymers may be useful in certain products. Illustrative are polyquaternium 4. polyquaternium 6, polyquaternium 7, polyquaternium 10, polyquaternium 11, polyquaternium 16, polyquaternium 22, and polyquaternium 32. Cationic polymers useful in the present invention include, but are not limited to, polyquaternium 4. polyquaternium 6, polyquaternium 7, polyquaternium 10, polyquaternium 11, polyquaternium 16, polyquaternium 22, polyquaternium 28, polyquaternium 32, and guar hydroxypropyltrimonium chloride. Preferred cationic polymers include POLYMER JR-125, POLYMER JR-400, Polymer JR-30M hydroxyethyl cellulosic polymers (polyquaternium 10) available from AMERCHOL: JAGUAR C13-S, guar hydroxypropyltrimonium chloride, available from Rhodia; and MERQUAT 100 and 280, a dimethyl dialkyl ammonium chloride (polyquaternium 6) available from Nalco. The cationic polymer when present may be in an amount of from greater than 0% to about 15%, preferably from about 0.5 to about 10% by weight, and more preferably from about 1 to about 5% by weight, based on the total weight of the composition.

Preservatives

Preservatives may be incorporated into the compositions to protect against the growth of potentially harmful microorganisms. Suitable traditional preservatives are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are methylchloroisothiazolinone and methyl isothiazolinone combinations, phenoxyethanol, methyl paraben, propyl paraben, urea, imidazolidinyl urea, sodium dehydroacetate, sodium benzoate, and benzyl alcohol. Preservatives may be employed in amounts ranging from 0.01%> to 2% by weight of the cosmetic composition.

Desquamation Agents

Desquamation agents may be present. Illustrative are the monocarboxylic acids. Monocarboxylic acids may be substituted or unsubstituted with a carbon chain length of up to 16. Particularly preferred carboxylic acids are the alpha-hydroxycarboxylic acids, beta-hydroxycarboxylic or polyhydroxycarboxylic acids. The term “acid” is meant to include not only the free acid but also salts and C₁-C₃o alkyl or aryl esters thereof and lactones generated from removal of water to form cyclic or linear lactone structures. Representative acids are glycolic, lactic, malic and tartaric acids. A representative salt that is particularly preferred is ammonium lactate. Salicylic acid is representative of the beta-hydroxycarboxylic acids. Amounts of these materials when present may range from 0.01 to 15% by weight of the cosmetic composition.

Preferred desquamation agents may be selected from the group consisting of glycolic acid, lactic acid, salicylic acid, retinoic acid, retinol and mixtures thereof, and including salt forms thereof.

Colorants

Colorants may either be dyes or pigments. A distinction is usually made between a pigment, which is insoluble in its vehicle (resulting in a suspension), and a dye, which either is itself a liquid or is soluble in its vehicle (resulting in a solution). A colorant can act as either a pigment or a dye depending on the vehicle involved. In some cases, a pigment can be manufactured from a dye by precipitating a soluble dye with a metallic salt. The resulting pigment is called a lake pigment.

Among the more common dyes are Alizarin, Azophloxin, Chrysoidin, Congo Red, Fuchsin acid, Gentian violet, Janus green, Methyl Red, Naphthol Green, Naphthol Yellow, Rose Bengal, Sudan II, Titan Yellow and combinations thereof. Amongst pigments, titanium dioxide and aluminum lakes (aluminum salts of organic dyes) are most common. Amounts of the colorant may, according to the type of cosmetic product range from 0.000001 to 10%, usually from 0.01 to 5% by weight of the cosmetic composition.

The compositions according to the instant disclosure may be prepared according to techniques that are well known to those skilled in the art, in particular those intended for the preparation of emulsions of oil-in-water or water-in-oil type. They may be in particular in the form of a simple or complex emulsion (O/W, W/O, O/W/O or W/O/W emulsion) such as a cream or a milk, in the form of a gel or a cream-gel, or in the form of a lotion.

Where the following terms are used in this specification, they are used as defined below. The terms “comprising,” “having,” and “including” are used in their open, non-limiting sense. The terms “a” and “the” are understood to encompass the plural as well as the singular. As used herein, the expression “at least one” means one or more and thus includes individual components as well as mixtures/combinations.

“Cosmetically acceptable” means that the item in question is compatible with any keratinous substrate. For example, “cosmetically acceptable carrier” means a carrier that is compatible with any keratinous substrate (e.g. hair or scalp).

A “physiologically acceptable medium” means a medium which is not toxic and can be applied to the skin, lips, hair, scalp, lashes, brows, nails or any other cutaneous region of the body. The composition of the instant disclosure may especially constitute a cosmetic or dermatological composition.

The phrase “essentially without” refers to less than or equal to 0.5, 0.1, 0.05 or 0.01 wt. %.

Except in the examples, or where otherwise explicitly indicated, all numbers in this description indicating amounts of material or conditions of reaction, physical properties of materials and/or use are to be understood as modified by the word “about.” All amounts are by weight of the compositions, unless otherwise specified.

It should be noted that in specifying any range of concentration or amount, any particular upper concentration can be associated with any particular lower concentration or amount. The disclosure of the invention as found herein is to be considered to cover all embodiments as found in the claims as being multiply dependent upon each other irrespective of the fact that claims may be found without multiple dependency or redundancy.

The instant disclosure will be better understood from the examples that follow, all of which are intended for illustrative purposes only and are not meant to limit the scope of the instant disclosure in any way.

### Example 1

| | |
|---|---|
| Ingredients (INCI Name) | % W/W |

Part A

| | |
|---|---|
| Deionized Water | 74 |
| Caffeine | 0.10 |
| Sodium Gluconate | 0.10 |
| Hydroxyethyl Acrylate/Sodium Acryloyldimethyl | 0.83 |
| Taurate Copolymer, Sorbitan Isostearate, Water | |
| Propanediol | 6.5 |
| Xanthan Gum | 0.15 |

Part B

| | |
|---|---|
| Biotin | 0.10 |
| Sodium Polyglutamate | 0.10 |
| Melatonin | 0.10 |

Part C

| | |
|---|---|
| Glycerin | 6.0 |
| Glycerin, Picea Mariana Bark Extract | 0.10 |
| Sodium Hyaluronate, Water, 1,2-Hexanediol | 1.0 |
| Water, Butylene Glycol, Dextran, Acetyl | 2.0 |
| Tetrapeptide-3, Red Clover Flower Extract | |
| Water, Glycerin, Pea Extract, Sodium Benzoate | 0.5 |
| Butylene Glycol, Water, Resveratrol | 0.10 |
| Benzyl Alcohol, Ethylhexylglycerin, Tocopherol | 1.0 |

Part D

| | |
|---|---|
| Deionized Water | 5.0 |
| Sodium Ascorbyl Phosphate | 0.10 |

Part E

| | |
|---|---|
| S-Bakuchiol | 0.50 |
| Polyglyceryl-4 Laurate/Sebacate, Polyglyceryl-6 | 1.5 |
| Caprylate/Caprate, Deionized Water | |

Part F

| | |
|---|---|
| Deionized Water | qs |
| Citric Acid | qs |
| | 100 |

The formulation under Example 1 was processed in the following manner. A 1500 mL beaker was charged with the water from Part A. Water in the beaker was stirred using an Ika dispersing blade operated from a Caframo mixer. Next, the caffeine (anhydrous powdered) was added into the beaker and mixed until uniformly dispersed. Subsequently, sodium gluconate was dosed into the beaker with continued stirring. Slowly Sepinov WEO (hydroxyethyl Acrylate/sodium Acryloyldimethyl Taurate Copolymer carried with aqueous sorbitan isostearate) was sprinkled with vortex formation into the beaker until thickening and a homogeneous mass resulted.

Propanediol and xanthan gum were premixed together and then charged to the main beaker with mixing until the mass was homogeneous. Part C premix was formed containing glycerin, Picea mariana Bark extract, sodium hyaluronate, 1,2-hexanediol, butylene glycol, dextran, Acetyl tetrapeptide-3, red clover flower extract, pea extract, sodium benzoate, resveratrol, benzyl alcohol, ethylhexyl glycerin, tocopherol and deionized water.

Part D was formed by mixing sodium ascorbyl phosphate in deionized water in a separate beaker followed by transfer to the main beaker. Part E was formed as a premix combining bakuchiol with polyglyceryl-4 laurate/sebacate, polyglyceryl-6 Caprylate/caprate in deionized water. Upon Premix E becoming uniform through mixing, Premix Part E was combined into the other ingredients in the main beaker. Adjustment of pH was done through addition of a citric acid water solution, in any necessary amount.

### Example 2

A series of bench and human clinical studies were conducted on the formula detailed under Example 1. These studies were:
(1) Assessment of efficacy through micro-camera image analysis.
(2) Assessment of reduction of hair loss by trichogram analysis.
(3) Evaluation of tensile properties of the hair fibers.

### Specifics of the Studies

Assessment of Efficacy Through Micro-Camera Image Analysis

Study Panel: 59 participants completed the study. The panel included both men and women, aged from 18 to 60 years.

Objective: The purpose of this study was to assess the efficacy of the investigational ‘Example 1’ (hereinafter the ‘formula’) in providing growth of new hair strands after 90 days of in-home use.

Experimental Design: Panelists were instructed to discontinue use of any cosmetic products on their hair and scalp 48 hours before beginning the study. Growth of new hair strands were evaluated by phototrichogram technique. The technique was performed by obtaining microimages of the scalp using a microcamera (I-Scope USB, Moritex, JP) with a 30× objective and polarized light. Measurements were done at the beginning and after 90 days of treatment.

Directions for Use: The formula was applied directly to damp or dry scalp while sectioning hair with dropper (pipette). Panelist applied up to 1-3 drops or enough to cover the entire scalp. Fingertip massage insured good penetration. No rinse allowed. Procedure was used once daily.

Assessment: Assessment was conducted by phototrichogram technique. During the period of in-home use, the panelists were instructed to fill out a Log of USE. Therein panelists recorded the days of application and any information/comments on the period of use.

Scalp imaging was performed using a micro-camera (i-Scope USB, Moritex, JP) with a 30× objective and polarized light. The micro-camera was positioned at a 90 degree angle to the scalp. Representative images were obtained of right and left parietal and temporal regions of each participating panelist. Nine microimages per participant were captured of the scalp in the initial condition and after 90 days of in-home use. Each image obtained corresponded to an area of 83.7 mm² on the scalp. These images were used to count the number of new hair strands and their density (number of hairs/cm²) before and after treatment.

Conclusion: Use of the formula provided a significant increase in density (fuller-looking) of new hair strands after 90 days in-home use.

Assessment of Hair Loss by Trichogram Analysis

Study Panel: 61 panelists completed this study. The panelists were split in two. Group 1 (30 people) used the example formula three days per week (baseline panelists). Group 2 (31 people) used the same formula but applied every day.

Objective: Purpose of this study was to assess efficacy of the formula to show improvement of the anagen/telogen ratio after 30 and 60 days of in-home use, in relation to the basal condition.

Experimental Design: The panelists were instructed to discontinue use of any cosmetic products on their hair/scalp for 48 hours prior to beginning the study. The assessment was done by trichogram exam. The exam involved collecting 50 to 100 hair strands for microscope analysis of the stage of biological growth cycle of the hair. This exam allowed assessment of the quantity of hair strands in the anagen and telogen phases. Evaluations were done at the beginning and after 30-days and then 60-days of daily treatment.

Directions for Use: The formula was applied directly to damp or dry scalp while sectioning hair with dropper (pipette). Panelist applied up to 1-3 drops or enough to cover the entire scalp. Fingertip massage insured good penetration. No rinse allowed. Procedure was used once daily.

Conclusion: The example formula achieved a significant increase in the anagen/telogen (A/T) ratio beginning at 30- and 60-days daily treatment. Mean A/T ratio for the baseline control was 3.8 and increased to 4.6 at 30-days. Mean A/T ratio rose to 5.1 at 60-days of once daily application. These results indicated there was significant variation in the number of hairs in the growing phase (anagen) and/or a reduction in number of hairs in the resting phase (telogen).

Of the everyday application group, 68% presented an increase in the anagen/telogen ratio after 30-days and 77% increase of the A/T ratio after 60-days daily use. Further, 19% of the everyday application group turned from telogen effluvium condition (alopecia) to normal condition after 60 days treatment.

Tensile Properties of Hair Fibers

Objective: The purpose of this study was to assess the mechanical resistance of the hair structure post treatment with the formula.

Experimental Design: Tresses of double bleached straight Caucasian hair were submitted to a control (10% sodium lauryl ether sulfate—‘SLES’) and to the formula treatments. After application of the treatments, 50 hair strands were assessed from the treated tresses. Each strand was held by a lower and upper claw connected to the load cell of a dynamometer along the upper part. The EMIC instrument was a model DL500 equipped with the dynamometer positioned with a 20N load cell.

The following parameters were measured: elongation at break, force at break, and Young's Modulus.

Conclusions: Formula 1 exhibited an enhanced efficacy in comparison to a shampoo (10% sodium lauryl ether sulfate) control. The enhanced efficacies were:
Strengthened hair
6% increased elasticity
44% resistance to hair breakage.

While the present compositions and methods have been described with reference to specific variations thereof, it should be understood by those skilled in the art that various changes may be made and equivalents may be substituted without departing from the true spirit and scope of the compositions and methods described herein. All patents and publications cited above are hereby incorporated by reference.

1. A topically applied scalp treatment composition comprising:
(i) about 0.001 to about 1% by weight of the composition of caffeine;
(ii) about 0.0001 to about 1% by weight of the composition of melatonin;
(iii) about 0.001 to about 3% by weight of the composition of a meroterpene;
(iv) about 0.1 to about 99% by weight of the composition of a cosmetically acceptable carrier, and wherein the treatment composition when applied to a scalp area induces an anagen to telogen ratio increase from a baseline control of about 3.8 compared to at least about 4.6 units of ratio after a 30-day daily application.

2. A composition according to claim 1 when applied to the scalp area induces an anagen to telogen ratio increase from a baseline control of about 3.5 compared to at least about 5.1 units of ratio after a 60-day daily application.

3. A composition according to claim 1 wherein the meroterpene is s-bakuchiol.

4. A composition according to claim 3 wherein the s-bakuchiol is present from about 0.01 to about 2% by weight of the composition.

5. A composition according to claim 3 wherein the s-bakuchiol is present from about 0.1 to about 1% by weight of the composition.

6. A composition according to claim 1 further comprising about 0.001 to about 1% by weight of the composition of resveratrol.

7. A composition according to claim 6 wherein the resveratrol is present from about 0.1 to about 0.5% by weight of the composition.

8. A composition according to claim 1 further comprising about 0.01 to 2% by weight of the composition of a red clover extract.

9. A composition according to claim 1 wherein the cosmetically acceptable carrier is water which has been treated to insure elimination of pathogenic microbes.

10. A composition according to claim 8 wherein the water has been treated in a manner selected from the group consisting of deionization, sterilization, pasteurization, and irradiation.

11. A method selected from the group consisting of promoting hair growth, slowing hair loss, thickening hair, protecting hair and scalp against stress induced degradation, and combinations thereof, comprising:
a) providing a composition comprising:
(i) about 0.001 to about 1% by weight of the composition of caffeine;
(ii) about 0.0001 to about 1% by weight of the composition of melatonin;
(iii) about 0.001 to about 3% by weight of the composition of a meroterpene; and
(iv) about 0.1 to about 99% by weight of the composition of a cosmetically acceptable carrier; and
b) topically applying the composition to hair and scalp, and wherein the treatment composition when applied to a scalp area induces an anagen to telogen ratio increase from a baseline control of about 3.8 compared to at least about 4.6 units of ratio after a 30-day daily application.

12. The method according to claim 11, wherein the treatment composition when applied to a scalp area induces an anagen to telogen ratio increase from a baseline control of about 3.5 compared to at least about 5.1 units of ratio after a 60-day daily application.

13. The method according to claim 10 wherein the meroterpene is s-bakuchiol.
